(19) United States
(12) Patent Application Publication
(10) Pub. No.: US 2013/0103091 A1
(43) Pub. Date: Apr. 25, 2013

(54) SPINAL FUSION INSTRUMENTATION AND SYSTEMS AND METHODS THEREOF

(71) Applicants: Frank Lugo ACOSTA, JR., Los Angeles, CA (US); Joseph Michael SAMPIETRO, Tarzana, CA (US)

(72) Inventors: Frank Lugo ACOSTA, JR., Los Angeles, CA (US); Joseph Michael SAMPIETRO, Tarzana, CA (US)

(21) Appl. No.: 13/277,783

(22) Filed: Oct. 20, 2011

(51) Int. Cl. A61B 17/70; A61B 17/88

(52) U.S. Cl. 606/259; 606/264; 606/279

(57) ABSTRACT

An instrumentation for use in spinal fusion surgery is disclosed. The instrumentation comprises a plurality of link segments configured to form a contoured shape that conforms to at least a section of vertebrae of a patient, and an interlocking mechanism configured to cause the plurality of link segments to maintain the contoured shape after the spinal fusion surgery.

### FIELD OF THE INVENTION

The present invention relates to systems and methods used in the performance of spinal correction procedures. More specifically, the present invention relates to spinal fusion instrumentation and systems and methods thereof.

### BACKGROUND

As depicted in FIG. 1A, the human spine (vertebral column) consists of twenty-four articulating vertebrae and nine fused vertebrae. The thirty-three total vertebrae in the vertebral column are split into five regions: cervical, thoracic, lumbar, sacral, and coccygeal. Each vertebrae is separated by intervertebral discs, which act as ligaments to hold the vertebrae together. Generally, the structure of a typical vertebra is comprised of a main vertebral body and a vertebral arch, which further comprises a pair of pedicles and a pair of laminae, enclosing a vertebral foramen and supporting seven processes—four articular (two each of a superior and an inferior process), two transverse, and one spinous. The main vertebral body is connected to the two pedicles, which are directed toward the posterior. The pedicles are each connected to the laminae, and from each of these junctions the superior articular processes and the inferior articular processes project upward and downward, respectively. The spinous process is directed obliquely downward, and extends from the junction of the two laminae. The transverse processes project from where the lamina joins the pedicle, between the superior and inferior articular processes. The body, pedicles and laminae define a vertebral foramen. The vertebral foramina, formed when the vertebrae are articulated to each other and extends from the first cervical vertebrae to the last lumbar vertebrae, houses the spinal cord and associated meninges. The cervical, thoracic, and lumbar regions are discussed briefly below.

The cervical region consists of seven vertebral bones and allow for movement of the neck and head. Cervical vertebrae (C1-C7) consist of a small body, pedicles directed laterally and toward the posterior, laminae, the articular processes (superior and inferior), and the transverse processes. Cervical vertebrae (C1-C7) are characterized by their smaller size and are easily distinguished by the presence of a foramen in each transverse process.

The thoracic region consists of twelve vertebral bones, with transverse processes that have surfaces that articulate with the ribs. The thoracic vertebrae (Th1-Th12) are distinguished by the facets present on the vertebral bodies that allow for articulation with the heads of the ribs, and the facets on the transverse processes of the first ten thoracic vertebrae that allow for articulation with the tubercles of the ribs. There is little normal motion of the vertebrae in the thoracic region, in comparison to the cervical and lumbar regions.

The lumbar region of the vertebral column consists of five vertebrae (L1-L5). They are the largest movable segments in the vertebral column, supporting more weight than the other vertebrae. Structurally, the vertebral body of each lumbar vertebra (L1-L5) is large, with strong pedicles, broad and short laminae, and a thick, broad spinous process.

Several spinal disorders affect the curvature of the vertebral column. For instance, degenerative disc disease, spinal disc herniation, fractures, tumors, or scoliosis all affect spinal curvature and can result in severe pain or neurological deficits. Also, spinal injury may affect the curvature, and require correction.

Correction of the spinal disorders mentioned above can be achieved via a spinal fusion procedure, a surgical technique that joins two or more vertebrae. Also, by correcting the spinal curvature can result in relief of pain caused by abnormal motion of the vertebrae.

One method of spinal fusion fuses the affected vertebrae by the grafting of bone tissue, either from the patient or a donor, using the patient's natural bone growth processes to fuse the vertebrae. Another method of spinal fusion implants an instrumentation into the vertebrae to support correction of spinal curvature, such instrumentation effectively fusing the vertebrae, or to encourage natural bone growth between the vertebrae.

Typical spinal fusion instrumentation comprises pedicle screws affixed to a support rod. Because human vertebrae have a natural contour as seen from FIG. 1A, it is often necessary to bend the support rod as depicted in FIG. 1B to make the rod conform to the natural contour. The surgical procedure involves implanting pedicle screws into the pedicles on one side of two adjacent vertebrae. The support rod is then bent to the proper contour by a surgeon, and then affixed to the protruding head portion of each of the pedicle screws. The same process is applied to the other side of the vertebrae.

There are unlimited degrees of freedom with which the rod may be bent when devising the contour. However, bending the support rod requires specialized equipment and the application of significant physical force to affect such bending, which can result in long procedure times and less than ideal contours. Because this is a very invasive surgery, involving large incisions and exposed bone tissue, there arises a need to perform the procedure efficiently and effectively, without sacrificing the structural integrity nor the degrees of freedom with which the instrumentation may be contoured.

### BRIEF SUMMARY OF THE INVENTION

In certain aspects, an instrumentation for use in a spinal fusion surgery is provided. The instrumentation can comprise a plurality of link segments configured to form a contoured shape that conforms to at least a section of vertebrae of a patient. The instrumentation can further comprise an interlocking mechanism configured to cause the plurality of link segments to maintain the contoured shape after the spinal fusion surgery.

In certain aspects, a system for use in a spinal fusion surgery is provided. The system can comprise a plurality of pedicle screws configured to be placed at vertebrae of a patient and a spinal instrumentation configured to be coupled to the plurality of pedicle screws and to form a contoured shape that conforms to at least a section of the vertebrae. The system can further comprise an interlocking mechanism configured to cause the plurality of link segments to maintain the contoured shape after the spinal fusion surgery.

In certain aspects, a method of performing a spinal fusion surgery is provided. The method can comprise providing a spinal fusion instrumentation comprising a plurality of link segments. The method can comprise placing a plurality of pedicle screws at vertebrae of a patient. The method can further comprise causing the plurality of link segments to form a contoured shape that conforms to at least a section of the vertebrae. The method can further comprise interlocking the plurality of link segments, thereby causing the plurality of link segments to maintain the contoured shape after the spinal fusion surgery. The method can further comprise affixing at least some of the plurality of link segments to the plurality of pedicle screws.

### BRIEF DESCRIPTION OF SEVERAL VIEWS OF THE DRAWINGS

FIG. 1A is a diagram depicting a side view of the human spine.

FIG. 1B is a diagram depicting a close up view of a spinal fusion device implanted on a spinal section during spinal fusion surgery.

FIG. 2A is a diagram depicting a first perspective view of an exemplary spinal fusion instrumentation comprising a plurality of hinge type link segments according to certain aspects of the present disclosure.

FIG. 2B is a diagram depicting a second perspective view of the exemplary spinal fusion instrumentation of FIG. 2A according to certain aspects of the present disclosure.

FIG. 3 is a diagram depicting a side view of one of the plurality of hinge type link segments shown in FIGS. 2A and 2B according to certain aspects of the present disclosure.

FIG. 4 is a diagram depicting a perspective view of a section of another exemplary spinal fusion instrumentation comprising a plurality of hinge type link segments according to certain aspects of the present disclosure.

FIG. 5A is a diagram depicting a side view of an exemplary spinal fusion instrumentation comprising a link assembly comprising a plurality of ball-and-socket type link segments according to certain aspects of the present disclosure.

FIG. 5B is a diagram depicting a cross-sectional view of the exemplary spinal fusion instrumentation of FIG. 5A, showing the link assembly and an internal cable assembly, according to certain aspects of the present disclosure.

FIG. 6 is a diagram depicting a side view of the internal cable assembly depicted in FIG. 5B separated from the rest of the exemplary spinal fusion instrumentation of FIG. 5B according to certain aspects of the present disclosure.

FIG. 7 is a diagram depicting a perspective view of the exemplary spinal fusion instrumentation of FIG. 5A coupled to a set of pedicle screws according to certain aspects of the present disclosure.

FIGS. 8A-D are diagrams depicting alternative views of an exemplary socket-type link segment used as certain link segments in the exemplary spinal fusion instrumentation 500 of FIGS. 5A and 5B.

FIGS. 9A-D are diagrams depicting alternative views of another exemplary socket-type link segment used as certain link segments in the exemplary spinal fusion instrumentation 500 of FIGS. 5A and 5B.

FIGS. 10A-D are diagrams depicting alternative views of an exemplary ball-type link segment used as certain link segments in the exemplary spinal fusion instrumentation 500 of FIGS. 5A and 5B.

FIGS. 11A-D are diagrams depicting alternative views of another exemplary ball-type link segment used as a link segment in the exemplary spinal fusion instrumentation 500 of FIGS. 5A and 5B.

FIGS. 12A-C are diagrams depicting alternative views of yet another exemplary ball-type link segment that can be used in place of other exemplary ball-type link segments depicted in FIGS. 10A-D and 11A-D.

FIGS. 13A-D are diagrams depicting alternative views of the hard stop comprising the internal cable assembly of FIG. 6.

FIGS. 14A-E are diagrams depicting alternative views of the male-threaded rod comprising the internal cable assembly of FIG. 6.

FIGS. 15A-D are diagrams depicting alternative views of the compression nut comprising the internal cable assembly of 6.

FIG. 16A is a diagram depicting yet another exemplary spinal fusion instrumentation according to certain aspects of the present disclosure.

FIG. 16B is a diagram depicting an internal structure of the exemplary spinal fusion instrumentation of FIG. 16A according to certain aspects of the present disclosure.

FIG. 17 is a flowchart illustrating an exemplary surgical method or process employing a spinal fusion instrumentation comprising a plurality of link segments according to certain aspects of the present disclosure.

### DETAILED DESCRIPTION

In the following detailed description, numerous specific details are set forth to provide a full understanding of various aspects of the subject disclosure. It will be apparent, however, to one ordinarily skilled in the art that various aspects of the subject disclosure may be practiced without some of these specific details. In other instances, well-known structures and techniques have not been shown in detail to avoid unnecessarily obscuring the subject disclosure.

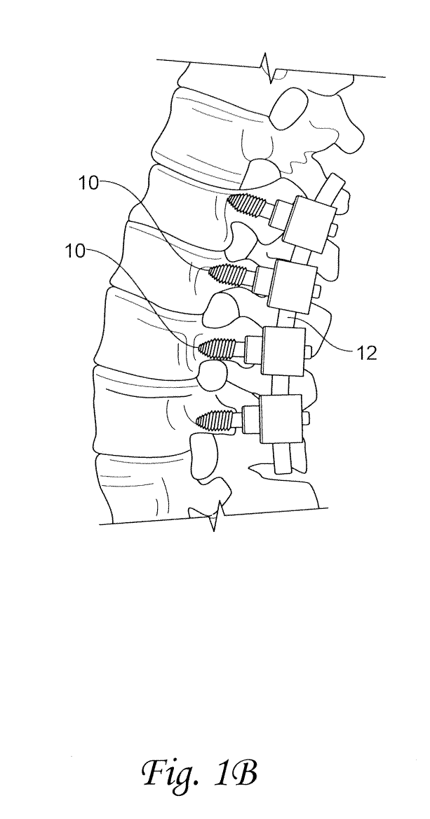

FIGS. 2A and 2B are diagrams depicting first and second perspective views of an exemplary spinal fusion instrumentation 200 comprising a plurality of hinge type link segments 210-270 according to certain aspects of the present disclosure. The plurality of link segments 210-270 are configured to form a contoured shape that conforms to at least a section of vertebrae of a patient during spinal fusion surgery. The plurality of link segments 210-270 can be made of a variety of materials including, but not limited to, a metal such as stainless steel, titanium, cobalt-chrome, or a polymer such as PEEK (polyetheretherketone).

As explained below, the spinal fusion instrumentation 200 further comprises an interlocking mechanism configured to cause the plurality of link segments 210-270 to maintain the contoured shape after the spinal fusion surgery. As best illustrated by FIG. 2B, the link segments (e.g., 210 and 220 and 230) include forked portions (e.g., 212 and 222 and 232) and flat portions (e.g., 214 and 224 and 234 of FIG. 2B).

As best illustrated by FIG. 2B, link segments in the exemplary spinal fusion instrumentation 200 are configured to be rotatably coupled to each other via hinge joints. For example, the first and second link segments 210 and 230 are coupled to each other via a first hinge joint 204 comprising the flat portion 214 of the first link segment 210 and the forked portion 222 of the second link segment 220. The second and third link segments 220 and 230 are coupled to each other via a second hinge joint 206 comprising the flat portion 224 of the second link segment 220 and the forked portion 232 of the third link segment 230.

In the exemplary spinal fusion instrumentation 200 of FIGS. 2A and 2B, the first hinge joint 204 provides a first degree of rotational freedom between the first and second link segments 210 and 220, and the second hinge joint 206 provides a second degree of rotational freedom between the second and third link segments 220 and 230. In particular, as illustrated in FIG. 2A, the first hinge joint 204 is configured to allow the first link segment 210 to rotate with respect to the second link segment 220 about a first rotation axis 205, and the second hinge joint 206 is configured to allow the second link segment 220 to rotate with respect to the third link segment 230 about a second rotation axis 207. This multi-degree rotational freedom permits the plurality of link segments 210-270 to form a contoured shape that conforms to vertebrae of a patient undergoing spinal fusion surgery.

As best illustrated by the link segment 270 shown in FIG. 2B, each flat portion (e.g., 274) of a link segment includes an opening (e.g., 275). As best illustrated by the link segment 210 shown in FIG. 2A, each forked portion (e.g., 212 of FIG. 2B) includes a two-pronged fork structure, each prong having an opening, namely, a first opening (e.g., 211) for a first (e.g., lower) prong and a second opening (e.g., 213) for a second (e.g. upper) prong. As illustrated in FIGS. 2A and 2B, the first opening 211 is configured to allow a screw 202 to pass through and the second opening 213 includes female threads formed therein to be threadedly engaged with the male threads of the screw 202. While FIGS. 2A and 2B show only one screw 202 for simplicity, it shall be understood by those skilled in the art that not shown are other screws configured to be threadedly engaged with female-threaded openings associated with other link segments (e.g., 220-270).

Such a threaded engagement between a screw (e.g., 202) and a female-threaded opening (e.g., 213) serves a number of purposes. For example, it serves to keep the plurality of link segments 210-270 connected to each other while allowing a surgeon or medical technician make angular adjustments between the plurality of link segments 210-270, thereby causing the link segments to form a contoured shape that conforms to at least a section of the patient's vertebrae being fused. For example, the screw 202 keeps the first link segment 210 connected to another link segment on the left (not shown) while angular adjustments are made between the link segments and a properly contoured shape is formed of the link segments. During this angular adjustment process, the screw 202 is only partially engaged with (e.g., not tightened to) the female-threaded opening 213. Furthermore, after all angular adjustments between the link segments are made, the threaded engagement is used as the spinal fusion instrumentation's interlocking mechanism causing the link segments 210-270 to maintain the contoured shape after the spinal fusion surgery. For example, after a desired contoured shape is attained, the surgeon can fully tighten the screw 202 to the female-threaded opening 213 and cause the link segments 210-270 to maintain the contoured shape after the spinal fusion surgery.

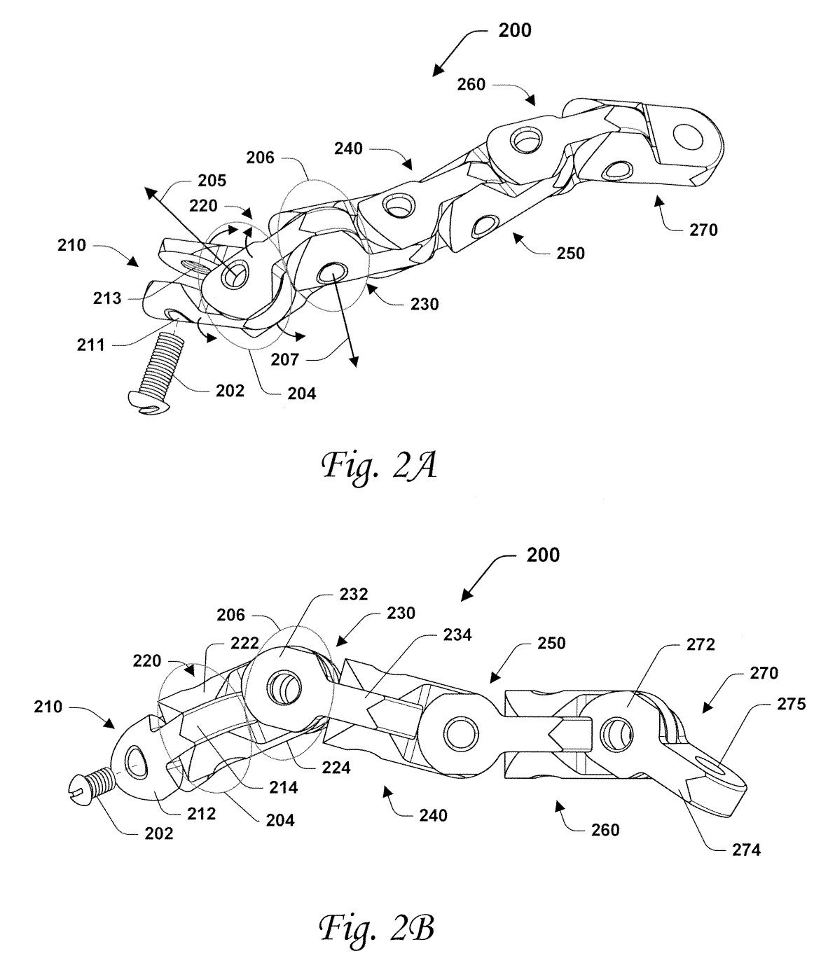

FIG. 3 is a diagram depicting a side view of one (e.g., the link segment 210) of the plurality of hinge type link segments 210-270 shown in FIGS. 2A and 2B. In particular, FIG. 3 depicts the forked portion 212 and the flat portion 214 of the link segment 210. As indicated above, the forked portion 212 comprises a two-pronged fork structure, each prong having an opening. The flat portion 214 is configured to be inserted in between the first and second prongs of the forked portion 222 of the second link segment 220 as best illustrated by FIG. 2B. The flat portion 214 of the first link segment 210 has a convex outer surface 215, and the forked portion 222 of the second link segment 222 has a corresponding concave internal surface (not shown). The convex outer surface 215 of the flat portion 212 the first link segment 210 and the concave inner surface of the forked portion 222 of the second link segment 220 are shaped and sized such that when the flat portion 214 is inserted into the forked portion 222, the opening in the flat portion 214 is aligned with the two openings in the forked portion 222, while allowing for an angular adjustment between the first and second link segments 210 and 220 through the hinge joint 206.

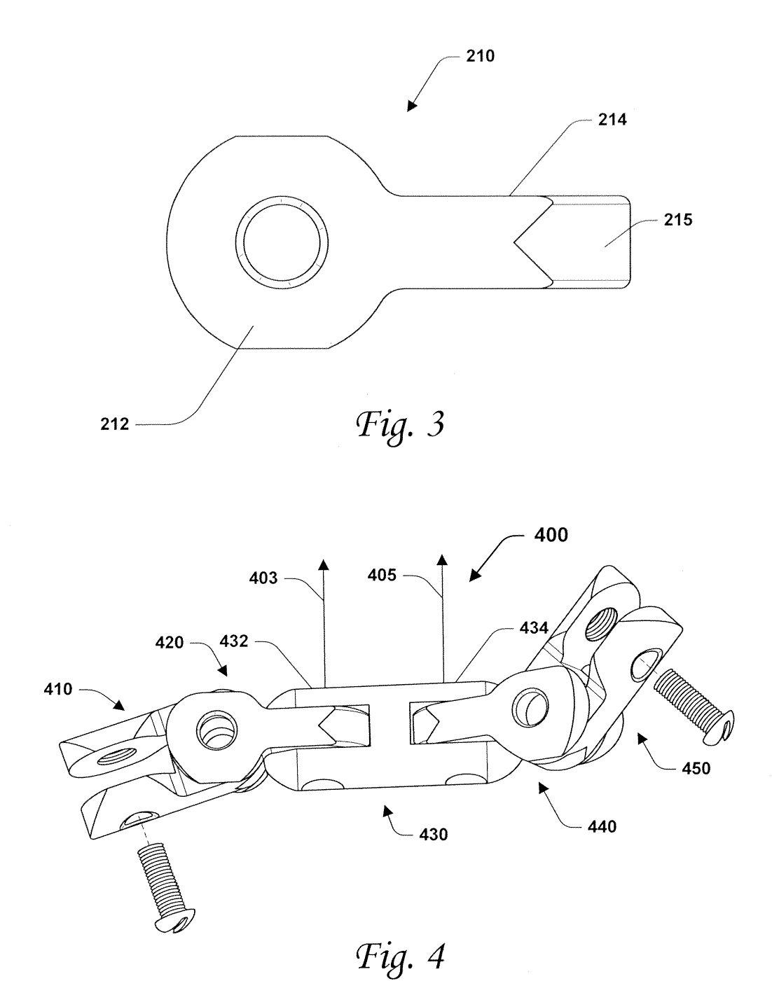

FIG. 4 is a diagram depicting a perspective view of a section of another exemplary spinal fusion instrumentation 400 comprising a plurality of hinge type link segments 410-450 according to certain aspects of the present disclosure. In the illustrated example, each of the hinge type link segments 410, 420, 440 and 450 includes a forked portion and a flat portion, substantially the same as the hinge type link segments 210-270 depicted in FIGS. 2A and 2B. The link segment 430, however, includes forked portions 432 and 434 on both ends instead of a forked portion and a flat portion as in the other link segments 410, 420, 440 and 450. Each of the forked portions 432 and 434 of the link segment 430 forms a hinge joint with a flat portion of the neighboring link segment 420 or 440 as shown in FIG. 4. In this arrangement, the second link segment 420 and the fourth link segment 440 are configured to rotate about the third link segment 430 along rotation axes 403 and 405 pointing towards the same direction.

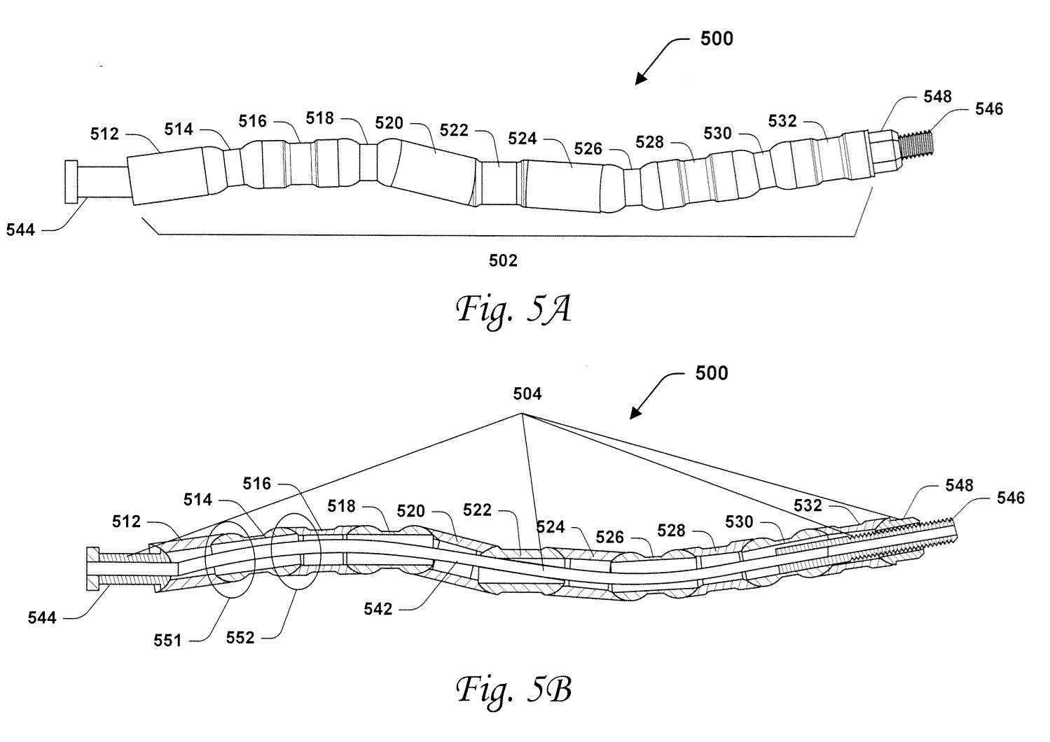

FIG. 5A is a diagram depicting a side view of an exemplary spinal fusion instrumentation 500 comprising a link assembly 502 comprising a plurality of ball-and-socket type link segments 512-532 according to certain aspects of the present disclosure. FIG. 5B is a diagram depicting a cross-sectional view of the exemplary spinal fusion instrumentation of FIG. 5A, showing the link assembly 502 and an internal cable assembly 504, according to certain aspects of the present disclosure. The plurality of ball-and-socket type link segments 512-532 can be made of a variety of materials including, but not limited to, a metal such as stainless steel, titanium, cobalt-chrome, or a polymer such as PEEK (polyetheretherketone).

The plurality of link segments 512-532 are configured to form a contoured shape that conforms to at least a section of vertebrae of a patient during spinal fusion surgery. As to be explained below, the spinal fusion instrumentation 500 further comprises an interlocking mechanism configured to cause the plurality of link segments 512-532 to maintain the contoured shape after the spinal fusion surgery.

In the exemplary spinal fusion instrumentation 500 of FIGS. 5A and 5B, the ball-and-socket type link segments 512-532 can be divided into two groups:

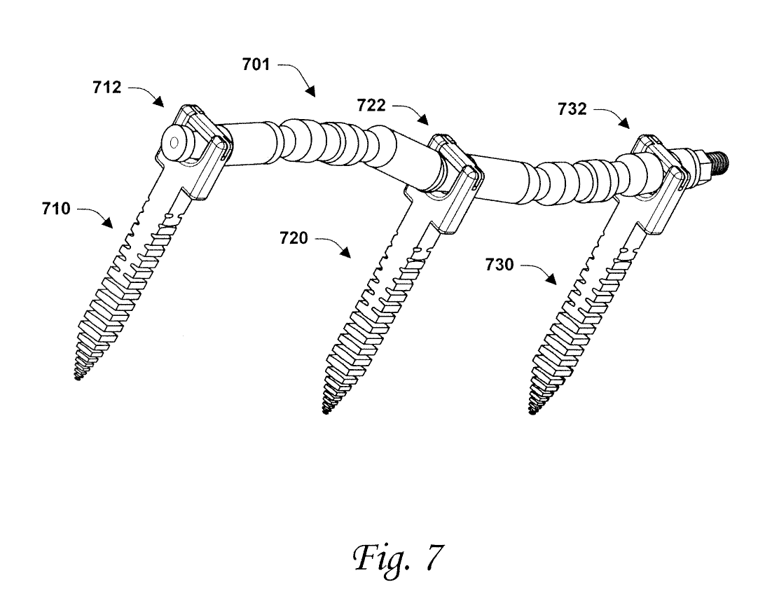

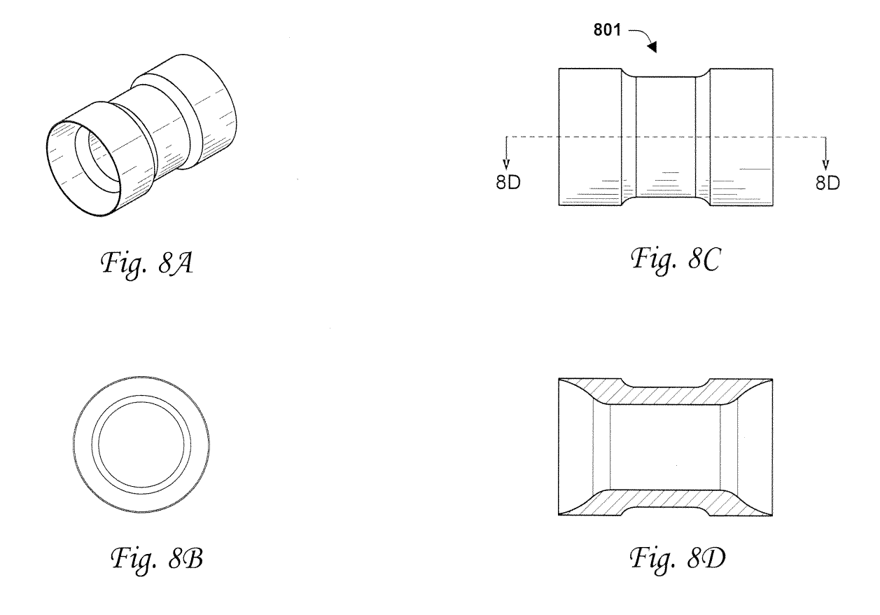

1) one group comprising socket-type link segments 512, 516, 520, 524, 528 and 532 having socket-shaped ends; and 2) another group comprising ball-type link segments 514, 518, 522, 526 and 530 having ball-shaped ends. FIGS. 8A-D are diagrams depicting alternative views of an exemplary socket-type link segment used as link segments 516, 528 and 532 in the exemplary spinal fusion instrumentation 500 of FIGS. 5A and 5B. This type of socket-type link segment includes an indented body portion 801 designed to facilitate coupling to a pedicle screw, for example. See FIG. 7. In certain embodiments, the socket-type link segment has a total length of 12.5 mm and an outer diameter of 8 mm. The indented body portion 801 has a length of 5.232 mm and an outer diameter of 7 mm. This socket-type link segment also includes socket-shaped ends at each end of the link segment. The socket-shaped ends are radiused on the internal surface of each end to receive a ball-type link segment as disclosed in the present application. This socket-type link segment also includes a bore having a diameter of 5 mm.

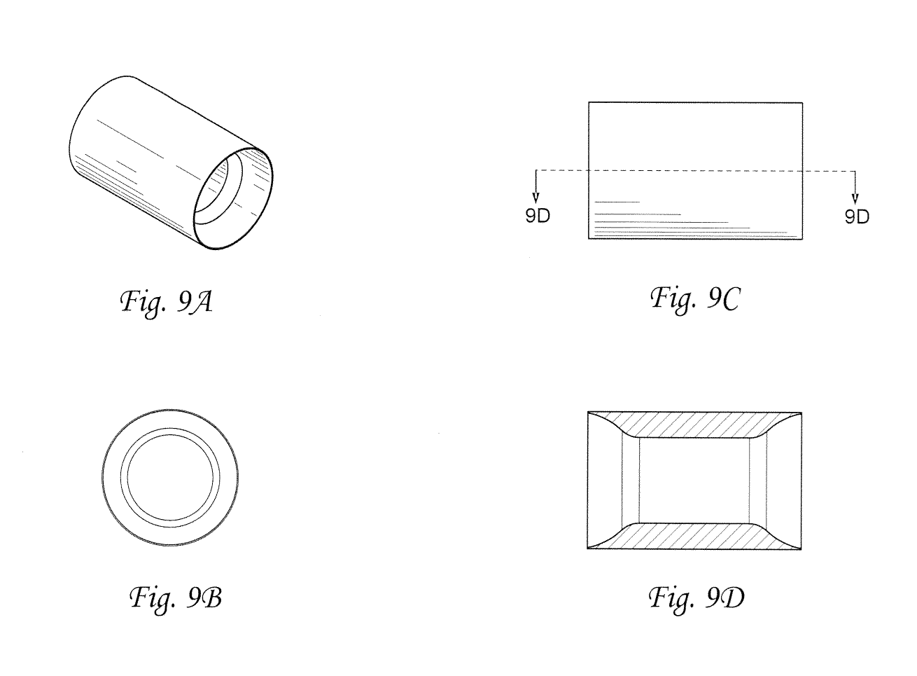

FIGS. 9A-D are diagrams depicting alternative views of another exemplary socket-type link segment used as link segments 512, 520 and 524 in the exemplary spinal fusion instrumentation 500 of FIGS. 5A and 5B. The socket-type link segment as depicted in FIGS. 9A-D has a total length of 12.5 mm and an outside diameter of 8 mm. This socket-type link segment also includes socket-shaped ends at each end of the link segment. The socket-shaped ends are radiused on the internal surface of each end to receive a ball-type link segment as disclosed in the present application. This socket-type link segment also includes a bore having a diameter of 5 mm.

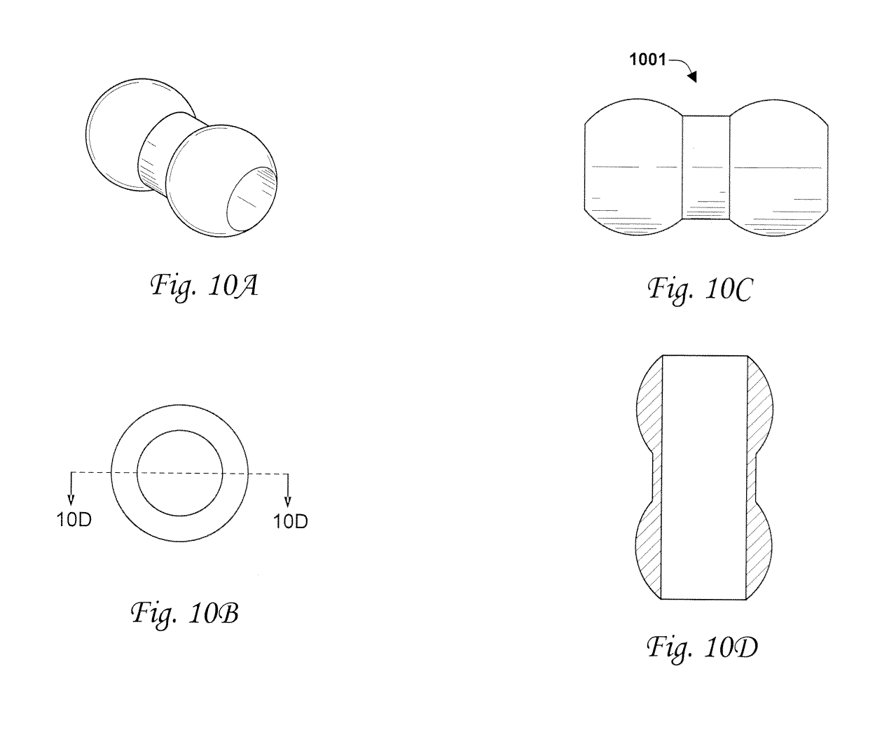

FIGS. 10A-D are diagrams depicting alternative views of an exemplary ball-type link segment used as link segments 514, 518, 526 and 530 in the exemplary spinal fusion instrumentation 500 of FIGS. 5A and 5B. This ball-type link segment includes an indented body portion 1001 designed to facilitate coupling to a pedicle screw, for example. See FIG. 7. In certain embodiments, the ball type link segment has a total length of 14.245 mm. The indented body portion 1001 has a length of 2.709 mm and an outside diameter of 6 mm. This ball-type link segment also includes ball-shaped ends that are radiused on the outer surface of each ball-shaped end of the link segment to be inserted into a socket-type link segment as disclosed in the present application. The ball-shaped ends of this ball-type link segment each have a length of 5.768 mm. This ball-type link segment also includes a bore having a diameter of 5 mm.

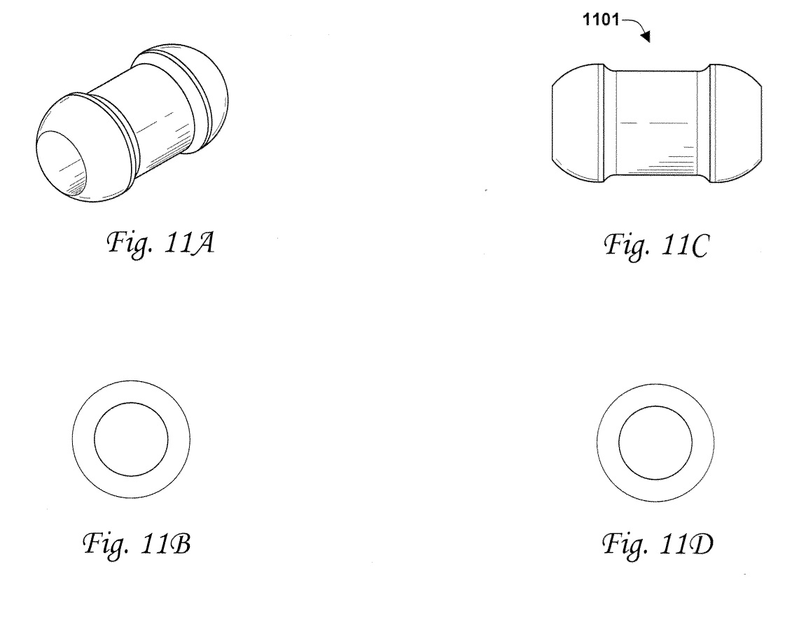

FIGS. 11A-D are diagrams depicting alternative views of another exemplary ball-type link segment used as link segment 522 in the exemplary spinal fusion instrumentation 500 of FIGS. 5A and 5B. This type of ball-type link segment includes an indented body portion 1101 designed to facilitate coupling to a pedicle screw, for example. See FIG. 7. In certain embodiments, this ball type link segment has a total length of 14.245 mm and an outside diameter of 8 mm. The indented body portion 1101 has an outside diameter of 7 mm. This ball-type link segment also includes ball-shaped ends that are radiused on the outer surface of each ball end of the link segment to be inserted into a socket-type link segment as disclosed in the present application. This ball-type link segment also includes a bore having a diameter of 5 mm.

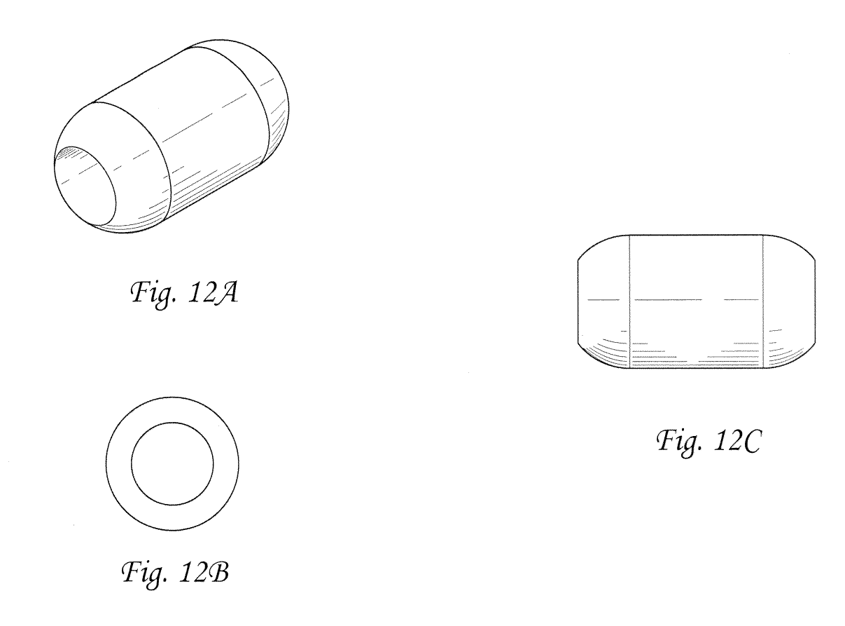

FIGS. 12A-C are diagrams depicting alternative views of yet another exemplary ball-type link segment that can be used in place of other exemplary ball-type link segments depicted in FIGS. 10A-D and 11A-D. In certain embodiments, this ball type link segment has a total length of 14.245 mm and an outside diameter of 8 mm. This ball-type link segment also includes ball-shaped ends that are radiused on the outer surface of each ball-shaped end of the link segment to be inserted into a socket-type link segment as disclosed in the present application. This ball-type link segment also includes a bore having a diameter of 5 mm.

As best illustrated in FIG. 5B, the socket-type link segment 512 is connected to the ball-type link segment 514 via a first ball-and-socket joint 551 comprising a socket-shaped end of the link segment 512 and a ball-shaped end of the link segment 514. Similarly, the ball-type link segment 514 is connected to the socket-type link segment 516 via a ball-and-socket joint 552 comprising a ball-shaped end of the link segment 514 and a socket-shaped end of the link segment 516. Such a ball-and-socket joint connection arrangement is repeated throughout the rest of the link assembly 502.

In the exemplary spinal fusion instrumentation 500 of FIGS. 5A and 5B, the ball-and-socket joint (e.g., 551 or 552) allows two consecutive link segments (e.g., the link segments 512 and 514 or the link segments 514 and 516) to rotate in a wide range of directions with respect to each other. This multi-degree rotational freedom permits the plurality of link segments 512-532 to form a contoured shape that conforms to the vertebrae of a patient during spinal fusion surgery. It shall be appreciated by those skilled in the art in view of the present disclosure that while a ball-and-socket joint is used to effectuate such a multi-degree rotational freedom in the exemplary spinal fusion instrumentation 500, other joint/connection arrangements may be used without departing from the scope of the present disclosure. For example, a universal joint comprising a pair of hinges located close together, oriented at 90° to each other, connected by a cross shaft, may be used in lieu of the ball-and-socket joint.

As best illustrated in FIG. 5B, the exemplary spinal fusion instrumentation 500, comprises a cable assembly 504 in addition to the plurality of ball-and-socket type link segments 512-532. The cable assembly 504 includes a cable 542 passing through the bores formed in the link segments 512-532, a hard stop 544 affixed to one end of the cable 542, a threaded rod 546 affixed to the other end of the cable 544, and a fastener 548 configured to be threadedly engaged with the threaded portion 546. In certain embodiments, the cable comprises a braided stainless steel cable having a diameter of in a range between about 1 and 3 mm, for example. In the illustrated example, the threaded portion 546 is a rod having a male-threaded portion, and the fastener 548 is a compression nut configured to be threadedly engaged with the male-threaded portion of the rod 546 and to compress the plurality of ball-and-socket type link segments 512-532 against the hard stop 544.

Alternatively, in some embodiments, a rod assembly comprising a thin flexible solid or semi-solid rod may be used in place of a cable assembly comprising a cable. In such embodiments, the rod assembly includes a rod configured to pass through bores formed in the plurality of link segments, hard stop affixed to one end of the rod, a threaded portion affixed to or formed in the other end of the rod, and a fastener configured to be threadedly engaged with the threaded portion. The solid or semi-solid rod can be made of a variety of materials including, but not limited to, a metal such as stainless steel, titanium, cobalt-chrome, a polymer such as PEEK (polyetheretherketone), or a fiber material. In case the rod has a circular cross-section, the diameter can be between about 1 and 2 mm, for example, depending on the rigidity of the material used. The solid or semi-solid rod is preferably flexible or malleable enough to allow for angular adjustments between the ball-and-socket type link segments 512-532, yet strong enough so that it would not deform under the force used to compress the link segments using the hard stop 544 and the fastener 548.

As the plurality of ball-and-socket type link segments 512-532 are compressed together between the compression nut 548 and the hard stop 544, the socket shaped ends of the socket-type link segments are compressedly engaged with the ball-shaped ends of the ball-type link segments owing to a difference in radii of curvature between the socket-shaped ends and the ball-shaped ends. For example, in one embodiment, the socket-shaped end has a radius of curvature of 4.10 mm, while the ball-shaped end has a radius of curvature of 4.00 mm. The compressed engagement between consecutive link segments (e.g., 512 and 513) provides an interlocking mechanism that causes the plurality of link segments 512-532 to maintain the contoured shape after the spinal fusion surgery by providing a rigidity between the link segments. The degree of rigidity may be adjusted by varying the difference in the radii of curvature, the material comprising the link segments (e.g., stainless steel versus titanium) or a combination of both.

The rigidity can be further enhanced by providing the ball-shaped end and the socket-shaped end with serrated surfaces designed to increase friction between the ends when they are compressedly engaged. In addition to or in lieu of the rigidity-enhancement methods discussed above, the respective surfaces of the ball-shaped and the socket-shaped end can be coated with a high-friction/anti-slip material such as polyurethane or vulcanized rubber to achieve a high coefficient of friction between the surfaces when they are compressed against each other. In addition to or in lieu of the rigidity-enhancement methods discussed above, the respective surfaces of the ball-shaped and the socket-shaped end can be impregnated with a pressure-sensitive adhesive material that solidifies and glues the surfaces together when the surfaces are compressed against each other.

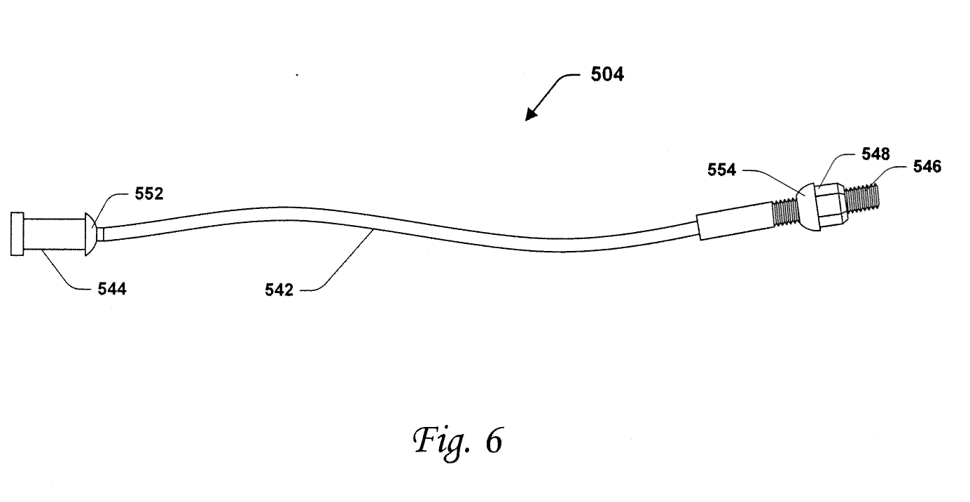

FIG. 6 is a diagram depicting a side view the cable assembly 504 depicted in FIG. 5B separated from the rest of the exemplary spinal fusion instrumentation 500. The hard stop 544 has a ball-shaped end 552 that is configured to couple to or engage with a socket-shaped end of the first link segment 512. Likewise, the compression nut 548 has a ball-shaped end 554 that is configured to couple to or engage with a socket-shaped end of the last link segment 532.

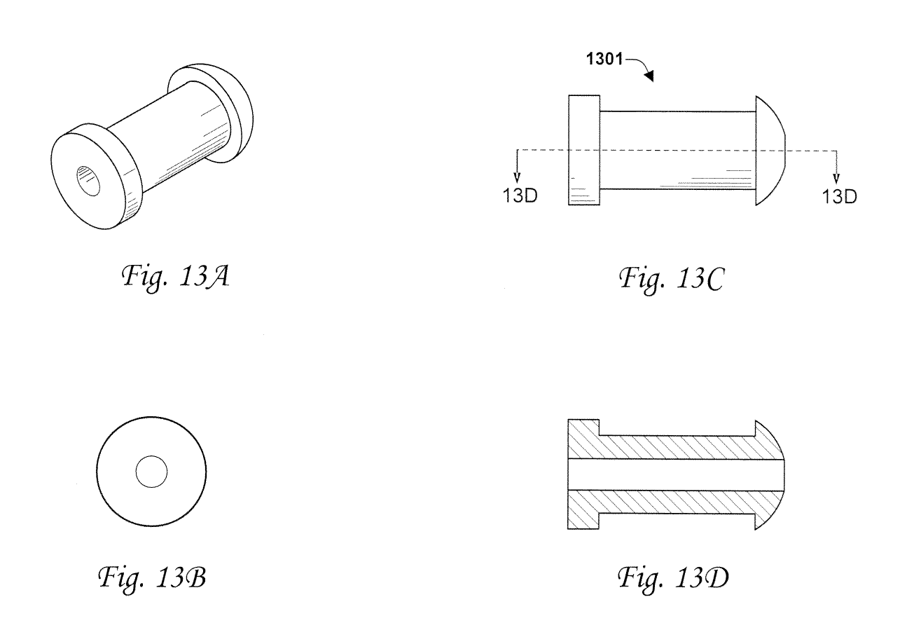

FIGS. 13A-D are diagrams depicting alternative views of the exemplary hard stop 544 used in the exemplary cable assembly 504 of FIG. 6. In the illustrated example, the hard stop 544 includes an indented body portion 1301 designed to facilitate coupling to a pedicle screw, for example. See FIG. 7. In certain embodiments, the hard stop 544 has a total length of 14 mm and an outside diameter of 7 mm. The indented body portion 1301 has a length of 10 mm and an outer diameter of 5 mm. The hard stop 544 also includes a bore with a diameter of 2.010 mm.

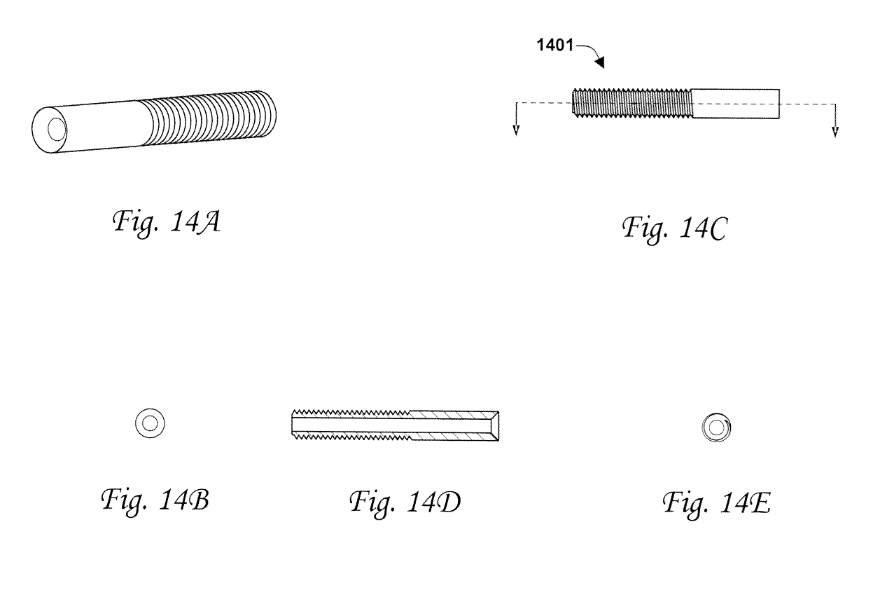

FIGS. 14A-E are diagrams depicting alternative views of the exemplary male-threaded rod 546 used in the exemplary cable assembly 504 of FIG. 6. This male-threaded rod 546 includes a threaded portion 1401 which is configured to be threadedly engaged with a fastener. In certain embodiments, the male-threaded rod 546 has a total length of 30 mm and an outside diameter of 4.250 mm. The threaded portion 1401 has a length of 17.663 mm and a thread diameter of 4.7 mm and a thread pitch of 1.05 mm. This male-threaded rod also includes a bore with a diameter of 2.010 mm.

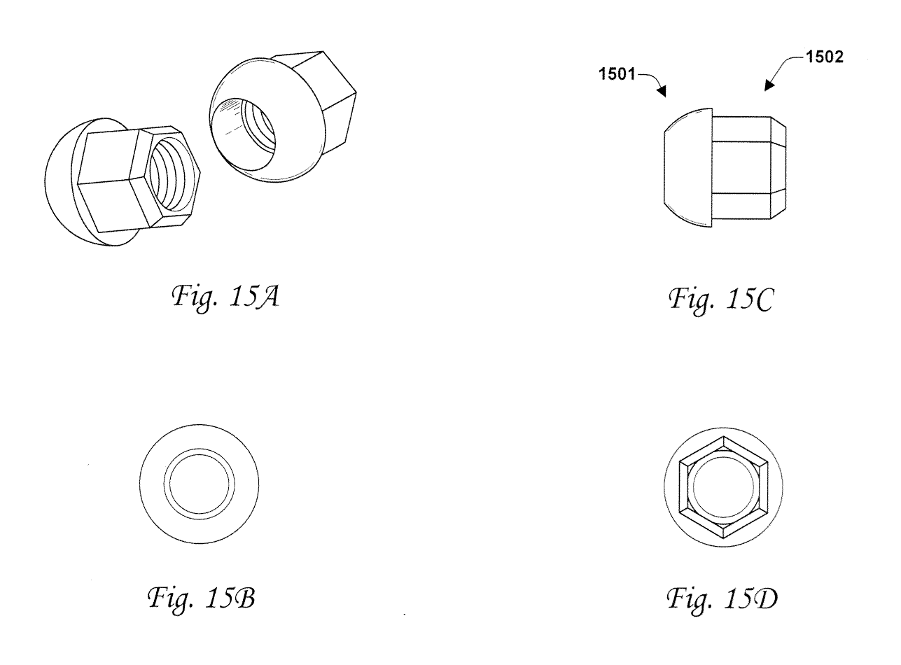

FIGS. 15A-D are diagrams depicting alternative views of the exemplary compression nut 548 used in the exemplary cable assembly 504 of FIG. 6. In certain embodiments, the compression nut 548 has a total length of 8.241 mm. A compression head 1501 has a length of 3.241 mm and is radiused for insertion into a socket-type link segment as disclosed in the present application. A hexagon-shaped nut end 1502 has a length of 5 mm, a polygonal diameter of 6.939 mm, and a width of 6 mm when measured at the centers of two opposing sides. The hexagon-shaped nut end 1502 also includes a bore, such bore having a thread diameter of 4.7 mm at the compression head 1501.

FIG. 7 is a perspective view of an exemplary spinal fusion instrumentation 701 coupled to a set of pedicle screws 710, 720 and 730 according to certain aspects of the present disclosure. For the purpose of illustration only without intent to limit the scope of the present disclosure in anyway, the spinal fusing instrumentation of FIG. 7 is a ball-and-socket type spinal fusion instrumentation, examples of which are described above with respect to FIGS. 5A and 5B. However, it shall be appreciated that other types of spinal fusion instrumentation having a plurality of link segments including the hinge type spinal fusion instrumentation of FIGS. 2A and 2B may be used in place. For ease of illustration, the pedicle screws 710, 720 and 730 are depicted without being implanted or embedded in vertebrae. During spinal fusion surgery, the spinal fusion instrumentation 701 is coupled to the pedicle screws 710, 720 and 730 after they are implanted or embedded in a patient's vertebrae.

In the illustrated example, the pedicle screws 710, 720 and 730 have respective retainer portions 712, 722, 732 that are configured to engage with indented portions (e.g., 801 of FIG. 8C) of the link segments comprising the spinal fusion instrumentation 710. The pedicle screws 710, 720 and 730 can be made of a variety of materials including, but not limited to, a metal such as stainless steel, titanium, cobalt-chrome, or a polymer such as PEEK (polyetheretherketone).

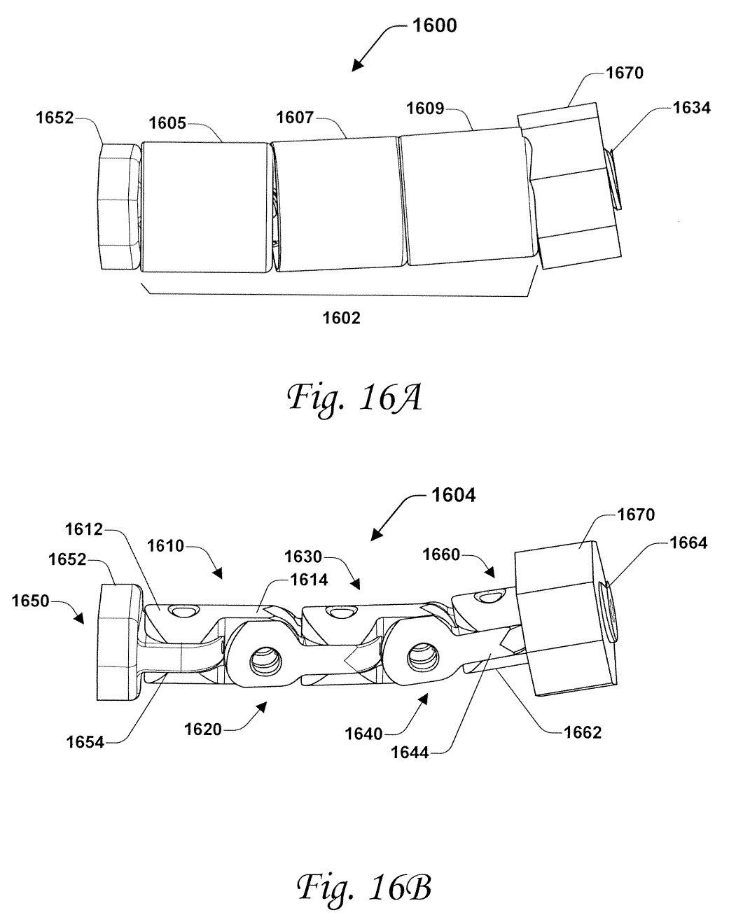

FIGS. 16A and 16B are diagrams depicting yet another exemplary spinal fusion instrumentation 1600 according to certain aspects of the present disclosure. The exemplary spinal fusion instrumentation 1600 comprises an outer link assembly 1602 comprising a plurality of tubular link segments 1605, 1607, 1609 and an inner link assembly 1604 comprising a plurality of hinge type link segments 1610, 1620, 1630 and 1640. The combination of the outer link assembly 1602 and the inner link assembly 1604 is configured to form a contoured shape that conforms to at least a section of vertebrae of a patient during a spinal fusion surgery. As described below, the exemplary spinal fusion instrumentation 1600 further comprises an interlocking mechanism configured to cause the combination of the outer link assembly 1602 and the inner link assembly 1604 to maintain the contoured shape after the spinal fusion surgery.

As indicated above, the outer link assembly 1602 comprises a plurality of tubular link segments 1605, 1607, 1609, and the inner link assembly 1604 comprises a plurality of hinge type link segments 1610-1640. The inner link assembly 1604 further comprises a hard stop 1650 comprising a stop portion 1652 and a flat link portion 1654 where the flat link portion 1654 is configured to be connected to a forked portion 1612 of the first inner link segment 1610 via a hinge joint. The inner link assembly 1604 further comprise a threaded rod 1660 having a forked link portion 1662 and a male-threaded portion 1664 where the forked link portion 1662 of the threaded rod 1660 is configured to be connected to a flat portion 1644 of the last inner link segment 1640. The inner link assembly 1604 further comprises a compression nut 1670 that is configured to be threadedly engaged with the male-threaded portion 1664 and further configured to compress the plurality of outer tubular link segments 1612-1616 against the stop portion 1652 of the hard stop 1650. The outer tubular link segments 1612-1616 are configured such that when they are compressed together, the outer tubular link segments form interlocking rigid joints through one or more interlocking mechanisms discussed with respect to FIGS. 5A and 5B. The rigid joints provide the interlocking mechanism whereby the combination of inner and outer link segments can maintain a contoured shape that conforms to a patient's vertebrae after the spinal fusion surgery.

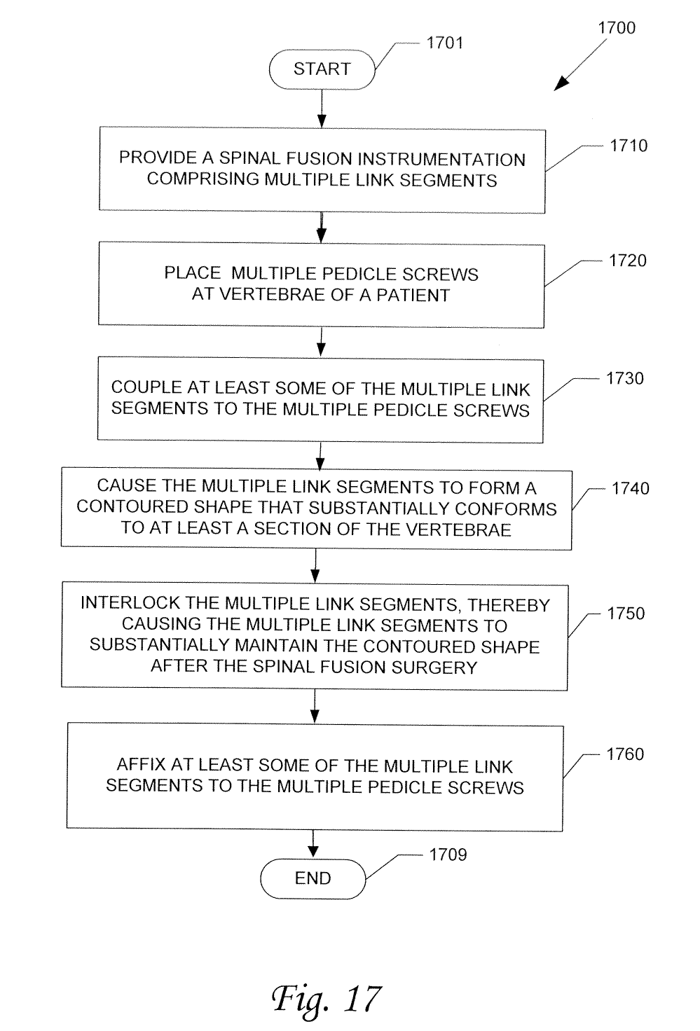

FIG. 17 is a flowchart illustrating an exemplary surgical method or process 1700 employing a spinal fusion instrumentation comprising a plurality of link segments according to certain aspects of the present disclosure. The exemplary process 1700 begins at start state 1701 and proceeds to operation 1710 in which a spinal fusion instrumentation comprising a plurality of link segments is provided. Non-limiting examples of such a spinal fusion instrumentation are described above with respect to FIGS. 2A and 2B, FIGS. 5A and 5B and FIGS. 16A and 16B.

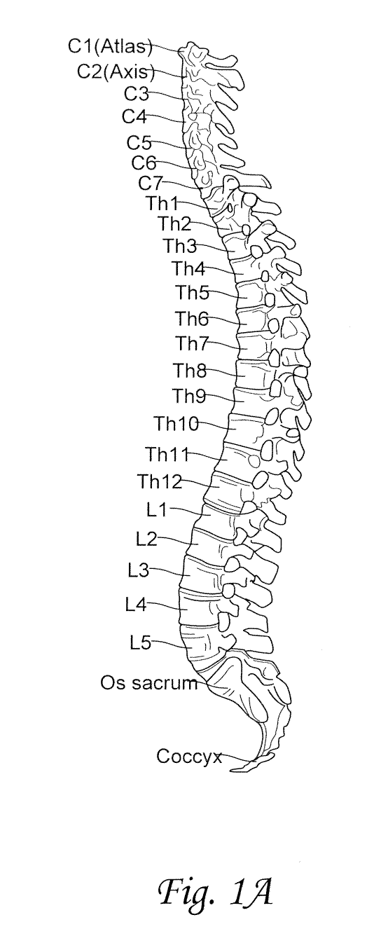

The exemplary process 1700 proceeds to operation 1720 in which a plurality of pedicle screws are placed at vertebrae of a patient. Non-limiting examples of pedicle screws are depicted in FIG. 1B and FIG. 7. The exemplary process 1700 proceeds to operation 1730 in which at least some of the plurality of link segments are coupled to the plurality of pedicle screws placed at the patient's vertebrae. This coupling allows for subsequent angular adjustments to the plurality of link segments described below.

At this stage, the link segments are not affixed to the pedicle screws, and the spinal fusion instrumentation's interlocking mechanism is not fully engaged. For example, in the case of the hinge type spinal fusion instrumentation 200 of FIGS. 2A and 2B, the screw (e.g., 211) is only partially engaged with the female-threaded opening (e.g., 213) in a forked end (e.g., 212) of a link segment (e.g., 210), thereby allowing for angular adjustments between link segments while keeping the link segments connected in a partially rigid manner. In the case of the ball-and-socket type spinal fusion instrumentation 500 of FIGS. 5A and 5B, the compression nut 548 is only partially compressing the plurality of link segments 512-532 against the hard stop 544, thereby allowing for angular adjustments between the link segments while keeping the link segments connected in a partially rigid manner.

The exemplary process 1700 proceeds to operation 1740 in which the plurality of link segments of the spinal fusion instrumentation are caused to form a contoured shaped that substantially conforms to at least a section of the vertebrae. Examples of link segments having contoured shapes are provided in FIGS. 2A and 2B and FIGS. 5A and 5B. This exemplary process 1740 can involve a surgeon or medical technician adjusting relative angles between the link segments to cause them to conform with a natural contour of the patient's vertebrae.

The exemplary process 1700 proceeds to operation 1750 in which the plurality of contoured link segments are interlocked with each other, thereby causing the plurality of contoured link segments to substantially maintain the contoured shape after the spinal fusion surgery. For example, in the case of the hinge type spinal fusion instrumentation 200 of FIGS. 2A and 2B, the screw (e.g., 211) is fully engaged with (e.g., tightened against) the female-threaded opening (e.g., 213). In the case of the ball-and-socket type spinal fusion instrumentation 500 of FIGS. 5A and 5B, the compression nut 548 is fully (e.g., tightly) compressing the plurality of link segments 512-532 against the hard stop 544.

The exemplary process 1700 proceeds to operation 1760 in which at least some of the link segments are affixed to the plurality of pedicle screws placed at the patient's vertebrae by the use of screws, retaining pins or other fastening mechanisms. The process terminates at end state 1709.

It shall be appreciated by those skilled in the art in view of the present disclosure that various described operations of the exemplary process 1700 may be performed in different orders. For example, in certain embodiments, the operation 1730 (coupling the link segments to the pedicle screws) may be performed after the operation 1740 (causing the link segments to form a contoured shape) or the operation 1750 (interlocking the contoured link segments). In such embodiments, the operation 1720 (placing the pedicle screws at the patient's vertebrae) may be performed after the operation 1740 or the operation 1750.

The description of the invention is provided to enable any person skilled in the art to practice the various embodiments described herein. While the present invention has been particularly described with reference to the various figures and embodiments, it should be understood that these are for illustration purposes only and should not be taken as limiting the scope of the invention.

There may be many other ways to implement the invention. Various functions and elements described herein may be partitioned differently from those shown without departing from the spirit and scope of the invention. Various modifications to these embodiments will be readily apparent to those skilled in the art, and generic principles defined herein may be applied to other embodiments. Thus, many changes and modifications may be made to the invention, by one having ordinary skill in the art, without departing from the spirit and scope of the invention.

A reference to an element in the singular is not intended to mean “one and only one” unless specifically stated, but rather “one or more.” The term “some” refers to one or more. Underlined and/or italicized headings and subheadings are used for convenience only, do not limit the invention, and are not referred to in connection with the interpretation of the description of the invention. All structural and functional equivalents to the elements of the various embodiments of the invention described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

We claim:

1. An instrumentation for use in a spinal fusion surgery, the instrumentation comprising:
a plurality of link segments configured to form a contoured shape that conforms to at least a section of vertebrae of a patient, and
an interlocking mechanism configured to cause the plurality of link segments to maintain the contoured shape after the spinal fusion surgery.

2. The instrumentation of claim 1, wherein the plurality of link segments comprise a first link segment and a second link segment disposed adjacent to the first link segment.

3. The instrumentation of claim 2, wherein the first link segment and the second link segment are configured to be rotatably coupled to each other via a first hinge joint.

4. The instrumentation of claim 3, wherein the first hinge joint comprises a first joint portion of the first link segment and a second joint portion of the second link segment.

5. The instrumentation of claim 2, wherein the interlocking mechanism comprises a screw threadedly engaged with the first hinge joint, the threaded engagement substantially preventing a rotation between the first and second link segments.

6. The instrumentation of claim 2, wherein the plurality of link segments further comprise a third link segment disposed adjacent to the second link segment and configured to be rotatably coupled to the second link segment via a second hinge joint.

7. The instrumentation of claim 6, wherein the first hinge joint provides a first degree of rotational freedom between the first and second link segments, and the second hinge joint provides a second degree of rotational freedom between the second link segment and the third link segment.

8. The instrumentation of claim 7, wherein the first hinge joint is configured to allow the first link segment to rotate with respect to the second link segment about a first rotation axis, and the second hinge joint is configured to allow the second link segment to rotate with respect to the third link segment about a second rotation axis.

9. The instrumentation of claim 2, wherein the first link segment and the second link segment are configured to be rotatably coupled to each other via a ball-and-socket joint.

10. The instrumentation of claim 9, wherein the ball-and-socket joint comprises a socket-shaped end of the first link segment and a ball-shaped end of the second link segment, the socket-shaped end being configured to receive the ball-shaped end.

11. The instrumentation of claim 10, wherein the interlocking mechanism comprises a compressive engagement between the ball-shaped end and the socket-shaped end.

12. The instrumentation of claim 10, further comprising a cable assembly, the cable assembly comprising a cable configured to pass through bores formed in the plurality of link segments, a hard stop affixed to one end of the cable, a threaded portion affixed to the other end of the cable, and a fastener configured to be threadedly engaged with the threaded portion.

13. The instrumentation of claim 12, wherein the threaded portion comprises a male-threaded rod, and the fastener comprises a nut configured to be threadedly engaged with the male-threaded rod and to compress the plurality of link segments against the hard stop.

14. The instrumentation of claim 10, further comprising a rod assembly, the rod assembly comprising a rod configured to pass through bores formed in the plurality of link segments, a hard stop affixed to one end of the rod, a threaded portion affixed to or formed in the other end of the rod, and a fastener configured to be threadedly engaged with the threaded portion.

15. The instrumentation of claim 10, wherein the ball-shaped end has a first radius of curvature, and the socket-shaped end has a second radius of curvature that is less than the first radius of curvature.

16. The instrumentation of claim 10, wherein the ball-shaped end and the socket-shaped end comprise serrated surfaces configured to increase a coefficient of friction between the surfaces.

17. The instrumentation of claim 10, wherein the ball-shaped end and the socket-shaped end comprise surfaces coated with a material configured to increase a coefficient of friction between the surfaces.

18. A system for use in a spinal fusion surgery, the system comprising:
a plurality of pedicle screws configured to be placed at vertebrae of a patient;
a spinal instrumentation configured to be coupled to the plurality of pedicle screws and to form a contoured shape that conforms to at least a section of the vertebrae; and
an interlocking mechanism configured to cause the plurality of link segments to maintain the contoured shape after the spinal fusion surgery.

19. The system of claim 18, wherein the plurality of link segments comprise a first link segment and a second link segment disposed adjacent to the first link segment, the first link segment and the second link segment being configured to be rotatably coupled to each other via a hinge joint.

20. The system of claim 19, wherein the plurality of link segments comprise a first link segment and a second link segment disposed adjacent to the first link segment, the first link segment and the second link segment being configured to be rotatably coupled to each other via a ball-and-socket joint.

21. A method of performing a spinal fusion surgery, the method comprising:
providing a spinal fusion instrumentation comprising a plurality of link segments;
placing a plurality of pedicle screws at vertebrae of a patient;
causing the plurality of link segments to form a contoured shape that conforms to at least a section of the vertebrae;
interlocking the plurality of link segments, thereby causing the plurality of link segments to maintain the contoured shape after the spinal fusion surgery; and
affixing at least some of the plurality of link segments to the plurality of pedicle screws.
